(19) United States
(12) Patent Application Publication
(10) Pub. No.: US 2024/0374282 A1
(43) Pub. Date: Nov. 14, 2024

(54) PUNCTURE DEVICE GUIDE FOR ULTRASOUND PROBE

(71) Applicant: CIVCO Medical Instruments Co., Inc., Kalona, IA (US)

(72) Inventors: Timothy Wayne Meder, Riverside, IA (US); Craig Joseph Cermak, Iowa City, IA (US); Matthew James Situmeang, Solon, IA (US); Jennifer Rae Steburg, North Liberty, IA (US); Michael Jay Lee, North Liberty, IA (US)

(21) Appl. No.: 18/646,418

(22) Filed: Apr. 25, 2024

Related U.S. Application Data
(60) Provisional application No. 63/500,708, filed on May 8, 2023.

(51) Int. Cl. A61B 17/34

(52) U.S. Cl. CPC A61B 17/3403; A61B 2017/3405; A61B 2017/3413

(57) ABSTRACT

A disposable puncture device guide for use with an ultrasound probe is described. The puncture device guide includes a fixed portion configured to attach directly or indirectly to the probe body and a deflection portion connected to the fixed portion by at least one living hinge. The deflection portion comprises at least one groove configured support a puncture device therein. Outward pressure on the at least one groove causes the deflection portion to rotate outwardly relative to the fixed portion to support the puncture device throughout a range of angles.

### CROSS-REFERENCE TO RELATED APPLICATION

This application claims priority under 35 U.S.C. § 119, based on U.S. Provisional Patent Application No. 63/500,708, filed May 8, 2023, titled “Needle Guide for Ultrasound Probe,” the disclosure of which is hereby incorporated by reference.

### BACKGROUND

This invention relates generally to medical devices and methods of use and more particularly to puncture device guide devices (e.g., needle guides) and methods of use with ultrasound transducers. It is a common medical practice to use a guide for releasable securement onto an ultrasound transducer to percutaneously guide a puncture device, such as a needle, cannula, trocar, or some other puncture device to some desired location within the body of a patient. The puncture device guide is arranged to enable the physician or other healthcare provider to guide the puncture device to a desired location within the body of the patient.

Because puncture device guides are used under sterile conditions, reusable puncture device guides must be sterilized and repackaged after each use. A disposable puncture device guide eliminates the expense and logistic effort of sterilization after each use. To be economically feasible, however, a disposable product must be of a simple design and easily produced at a low cost. The present design achieves these objectives by greatly simplifying mounting the device to the ultrasound probe and by the puncture device guide itself being made as a single component. However, embodiments consistent with implementations described herein may also be formed using a multi-part construction.

### BRIEF DESCRIPTION OF THE DRAWINGS

FIG. 1 is an isometric view illustrating one embodiment of a puncture device guidance device installed on an ultrasound probe, consistent with embodiments described herein;

FIG. 2 is another isometric view of puncture device guide of FIG. 1 illustrating a deflected or flexed orientation;

FIG. 3 is still another isometric view of the puncture device guide of FIG. 1 illustrating additional detail;

FIG. 4 is an isometric view of a mounting sheet positioned on an ultrasound probe for affixing the puncture device guide of FIG. 1;

FIGS. 5, 6 and 7 are isometric, side, and top views, respectively, of a further embodiment of a puncture device guide and mounting sheet consistent with implementations described herein;

FIGS. 8, 9, 10, and 11 are front isometric, rear isometric, bottom, and front views, respectively, of a yet another embodiment of a puncture device guide consistent with implementations described herein;

FIGS. 12, 13, and 14 are cross-sectional views taken along the lines A-A, B-B, and C-C in FIG. 11, respectively;

FIGS. 15A, 15B, and 15C are front isometric, front, and bottom views, respectfully, of another embodiment of a mounting plate for use in securing the puncture device guide of FIG. 8;

FIGS. 16A and 16B are front isometric views of the mounting plate of FIGS. 15A-15C installed onto a probe cover, consistent with implementations described herein;

FIG. 17 is a front isometric view of the mounting sleeve of FIGS. 15A-15C installed onto an ultrasound probe interface pad, consistent with implementations described herein;

FIGS. 18, 19, 20, and 21 are front isometric, rear isometric, bottom, and front views, respectively, of a yet another embodiment of a puncture device guide consistent with implementations described herein;

FIGS. 22, 23, and 24 are cross-sectional views taken along the lines A-A, B-B, and C-C in FIG. 21, respectively;

FIGS. 25A, 25B, and 25C are front isometric, front, and bottom views, respectfully, of another embodiment of a mounting structure for use in securing the puncture device guide of FIG. 18;

FIG. 26A and 26B are front isometric views of the mounting structure of FIGS. 25A-25C installed onto a probe cover, consistent with implementations described herein; and

FIGS. 27A and 27B are front isometric views of the mounting structure of FIGS. 15A-15C installed onto an ultrasound probe interface pad, consistent with implementations described herein.

### DETAILED DESCRIPTION OF THE PREFERRED EMBODIMENTS

The following detailed description refers to the accompanying drawings. The same reference numbers in different drawings may identify the same or similar elements. Also, the following detailed description does not limit the invention.

Implementations described herein relate to guidance devices for facilitating the placement of a puncture device (e.g., a needle) at a defined position relative to an ultrasound probe.

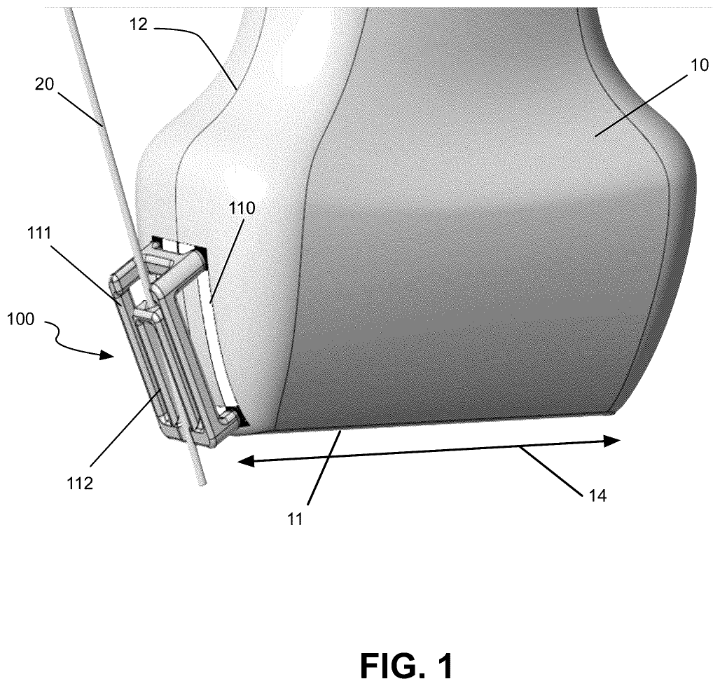

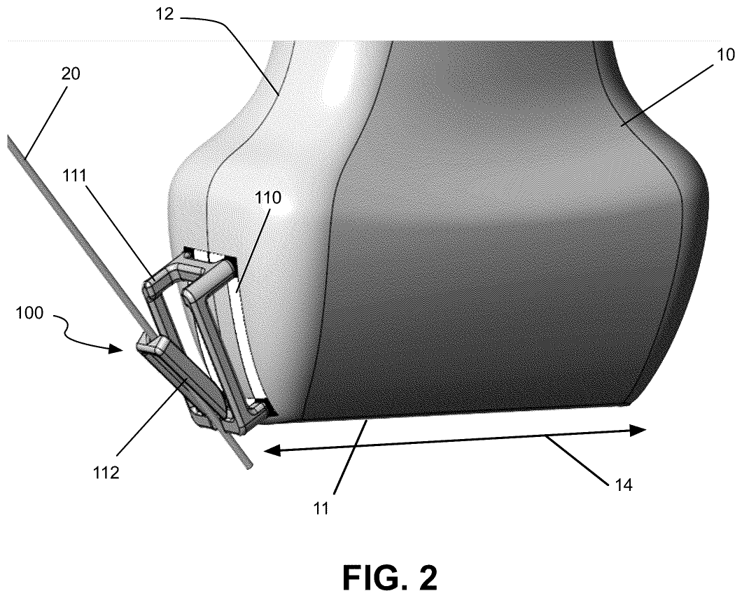

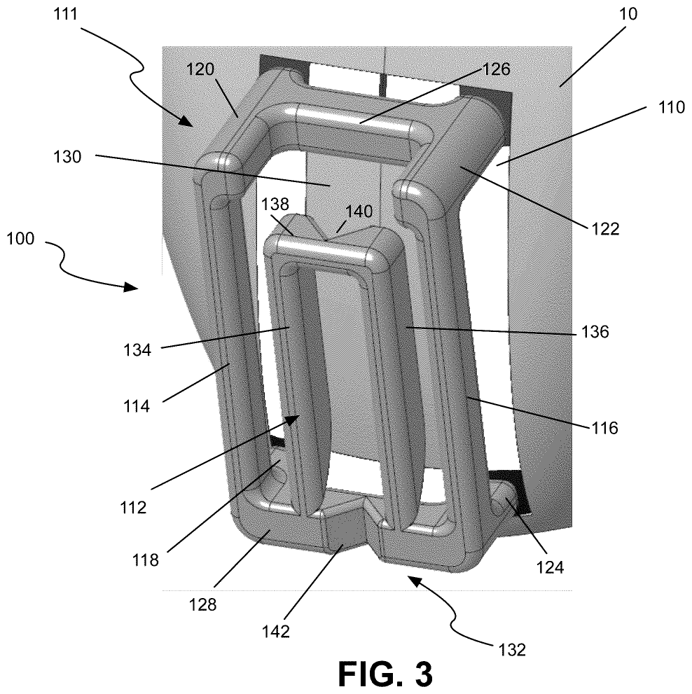

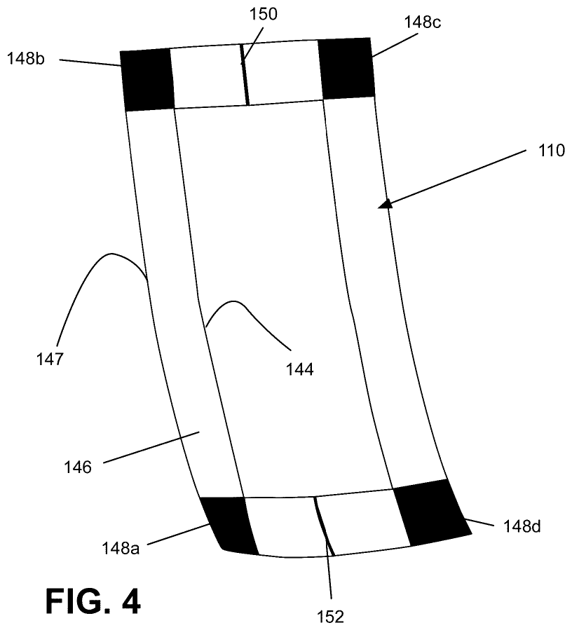

FIG. 1 is an isometric view of a puncture device guide 100 consistent with implementations described herein mounted on an ultrasound probe 10. FIG. 2 is another isometric view of puncture device guide 100 illustrating a deflected or flexed orientation. FIG. 3 is still another isometric view of puncture device guide 100 illustrating additional detail. FIG. 4 is an isometric view of a mounting sheet 110 positioned on ultrasound probe 10 for affixing puncture device guide 100 to probe 10.

As shown, puncture device guide 100 includes a fixed portion 111 and a deflection portion 112 for supporting a needle or other puncture device (e.g., needle 20 in FIGS. 1 and 2) in a range of angular orientations relative to fixed portion 111. As shown in FIG. 3, fixed portion 111 includes a frame-like configuration having a pair of side rails 114 and 116, four legs 118, 120, 122, and 124, which project perpendicularly from side rails 114 and 116, and cross members 126 and 128. In one implementation, as shown in FIG. 3, proximal cross member 126 may connect legs 120 and 122 and distal cross member 128 may connect the distal ends of side rails 114 and 116. As described below, such a configuration provides a space 130 between legs 120 and 122 and between side rails 114 and 116 for accommodating the receipt of needle 20 therein. As shown in the figures, legs 118-124 each include free ends that engage and attach to a surface of ultrasound probe 10, as described in additional detail below.

Consistent with implementations described herein, distal cross member 128 may form the base of a living hinge or spring hinge component 132 for coupling deflection portion 112 to fixed portion 111. As shown in FIG. 3, deflection portion comprises a pair of deflection side rails 134 and 136 that project proximally from distal cross member 128 parallel to side rails 114 and 116. A cross member 138 connects the proximal ends of deflection side rails 134 and 136. In some implementations, deflection side rails 134 and 136 have a length shorter than that of side rails 114 and 116, so that deflection portion 112 is completely contained within fixed portion 111.

As shown in FIG. 3, an inward facing groove 140 is formed in a central portion of cross member 138 and an outward facing groove 142 is formed in a portion of distal cross member 128 between deflection side rails 134 and 136. Grooves 140 and 142 are sized to accommodate a variety of needle gauge sizes. For example, one or both of grooves 140 and 142 may be v-shaped grooves (commonly referred to as “v-grooves”). In other implementations, grooves 140/142 may have a different configuration, such as a cylindrical, slotted, etc.

Consistent with implementations described herein, puncture device guide 100 may be formed of a suitably resilient or flexible material, such as a plastic or polymeric material and have a thickness or overall dimensions in a region of living hinge component 132 and/or deflection portion 112 that enable at least a portion of living hinge component 132 and/or deflection portion 112 to flex or rotate upon exertion of a rotational load.

As shown in FIGS. 1 and 2, during use, needle 20 may be inserted into grooves 140 and 142. To achieve a desired needle angle, an outward pivotal force may be applied to a proximal end of needle 20. This force causes needle 20 to urge cross member 138 outwardly relative to side rails 114 and 116, which causes side rails 134 and 136 to rotationally deflect or deform relative to distal cross member 128. Such a one-piece construction may be referred to as a “living hinge” given that there are no discrete and independently movable hinge components or mechanisms.

Furthermore, consistent with some implementations, deflection side rails 134/136 and/or cross member 128 of living hinge component 132 may be formed to provide a neutral bias to deflection portion 112. In this manner, although deflection portion 112 may be rotated upon exertion of pressure by needle 20 on inward facing groove 140, the dimensions and thicknesses of the relative components may be such that deflection portion 112 may be biased to return to its neutral position, thus maintaining a pressure on needle 20 throughout its range of motion.

As shown, the two grooves 140 and 142 are configured to guide needle 20 along the scan plane 14 of the probe 10. The grooves are oriented in opposing directions so that as pressure is applied by the needle 20 to rotate the deflection portion 112 about the live hinge to adjust the angle with respect to the fixed portion 111 of the guide, the needle 20 is forced into both grooves simultaneously. Grooves 140/142 allow the puncture device guide to work with various sizes of needles and hold them in the scan plane of the ultrasound probe while allowing the physician full control of the needle angle within the scan plane. FIG. 1 illustrates a needle 20 positioned within grooves 140/142 and oriented in a first (e.g., neutral or unflexed) angular position. FIG. 2 illustrates needle guide 100 and probe 10 with the deflection portion 112 flexed to allow the needle 20 to present at a more acute angle with respect to the probe face 11.

According to embodiments described herein, puncture device guide 100 may be formed as a single plastic or similarly flexible or resilient part. Exemplary manufacturing processes include injection molding the part out of acetal (polyoxymethylene), polypropylene, or any other suitable plastic material.

As shown in the figures, in one implementation, puncture device guide 100 may be attached to ultrasound probe 10 subsequent to manufacture of probe 10 and does not require configuration of ultrasound probe 10 to accommodate the attachment of puncture device guide 100. In one implementation, puncture device guide 100 is attached via adhesive mounting sheet 110. As shown in FIG. 4, mounting sheet 110 is sized similarly to a periphery of puncture device guide 100 and may include a back face 144 and a front face 146. To affix puncture device guide 100 to ultrasound probe 10, mounting sheet 110 may include an adhesive layer 147 on back face 144 and adhesive pads 148a-148d in four positions on front face 146 that correspond to the free ends of legs 118-124 on puncture device guide 100.

In the embodiment shown, front face 146 is generally white with black markings in the four corners 148a-148d. Other colors may be used, or the adhesive film may be clear with just the corners and alignment stripes marked. Adhesive layer 147 on back face 144 may be on the full face or may only be placed in the four marked positions. In addition to the markings for the four corners, the mounting sheet 110 may include two alignment stripes 150, 152, which are used to allow the user to align the mounting sheet 110 to a centerline 12 on the ultrasound probe 10 which corresponds to the scan plane of the probe. In this implementation, release layers (not shown) are initially provided on adhesive layer 147 and adhesive pads 148a-148d. Prior to use of puncture device guide 100, the release layers are removed and mounting sheet 110 is affixed to ultrasound probe 10 via adhesive layer 147 using alignment stripes 150/152. Free ends of legs 118-124 of puncture device guide 100 are then affixed to adhesive pads 148a-148d to secure puncture device guide 100 to probe 10.

In a further embodiment of the invention (not shown), puncture device guide 100 may accommodate use of a probe cover or sheath. In this embodiment, free ends of legs 118-124 may be provided with adhesive pads thereon. In this configuration, the mounting sheet 110 is attached to the probe 10 and a transparent or translucent probe cover is installed over the probe 10 and over the mounting sheet 110. Mounting sheet 110 adheres to the probe cover. Subsequently, the adhesive pads on legs 118-124 then adheres the puncture device guide 100 to the probe cover directly over the adhesive pads 148a-148d.

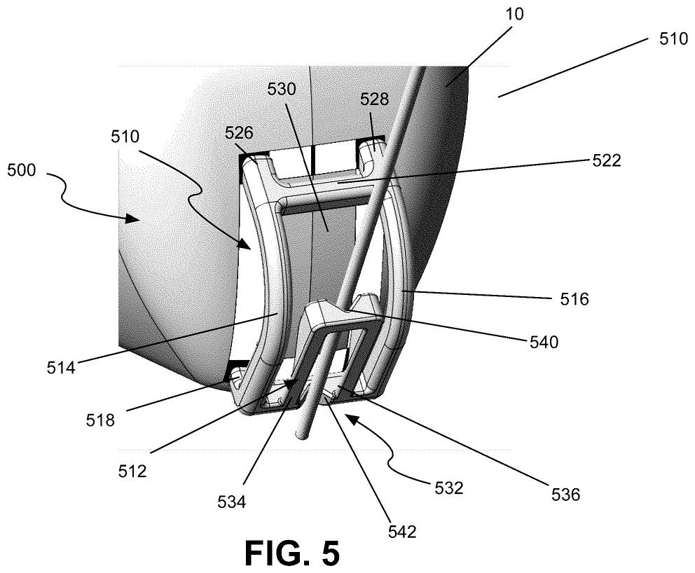

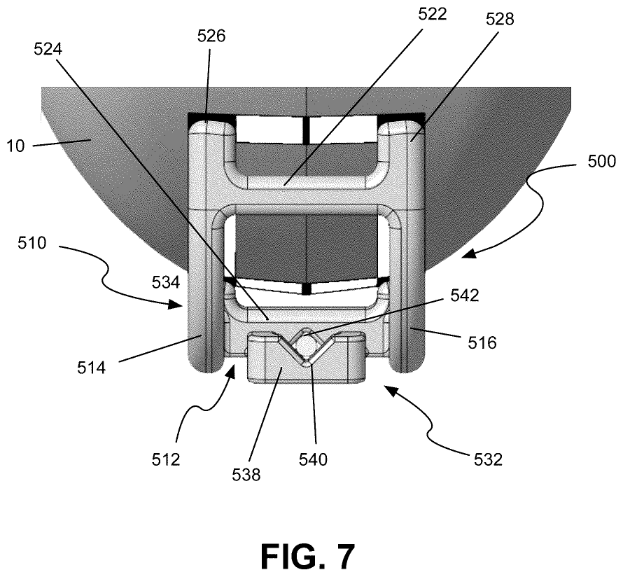

FIGS. 5, 6 and 7 illustrate isometric, side, and top views, respectively, of a further embodiment of a puncture device guide 500 and mounting sheet 110 mounted on an ultrasound probe 10.

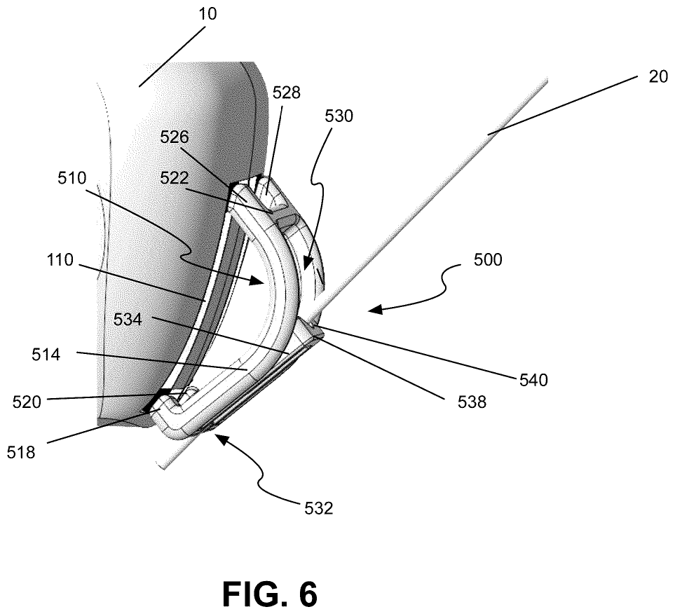

As shown, puncture device guide 500 includes a fixed portion 510 and a deflection portion 512 for supporting a needle or other puncture device (e.g., needle 20 in FIGS. 5 and 6) in a range of angular orientations relative to fixed portion 510. Consistent with the illustrated embodiment, fixed portion 510 includes a frame-like configuration having a pair of curved side rails 514 and 516, distal legs 518 and 520, which project perpendicularly from a distal portion of side rails 514 and 516, and cross members 522 and 524. As shown in FIGS. 5 and 6, rather than including perpendicularly projecting proximal legs, as provided in the embodiment of FIGS. 1-3, curved side rails 514/516 of puncture device guide 500 include downwardly curving proximal portions 526 and 528. Proximal portions 526/528 and distal legs 518/520 each include free ends which engage mounting sheet 110 (or the probe cover), as described above.

As shown in FIG. 5, proximal cross member 522 connects curving proximal portions 526 and 528 and distal cross member 524 connects the distal ends of side rails 514 and 516. As described below, such a configuration provides a space 530 between curving proximal portions 526 and 528 for accommodating the receipt of needle 20 therein. Similar to the embodiment of FIGS. 1-4, described above, during use free ends of legs 518/520 and proximal portions 526/528 of puncture device guide 500 are affixed to adhesive pads 148a-148d in mounting sheet 110 (or to a probe cover positioned over such pad) to secure puncture device guide 500 to probe 10.

Distal cross member 524 may form the base of a living hinge component 532 for coupling deflection portion 512 to fixed portion 510. As shown in FIG. 5, deflection portion 512 comprises a pair of deflection side rails 534 and 536 that project proximally from distal cross member 524 parallel to side rails 514 and 516. A cross member 538 connects the proximal ends of deflection side rails 534 and 536. In some implementations, deflection side rails 534 and 536 have a length shorter than that of side rails 514 and 516, so that deflection portion 512 is completely contained within fixed portion 510.

Consistent with implementations described herein, puncture device guide 500 may be formed of a suitably resilient or flexible material, such as a plastic or polymeric material and have a thickness or overall dimensions in a region of living hinge component 532 and/or deflection portion 512 that enable at least a portion of living hinge component 532 and/or deflection portion 512 to flex or rotate upon exertion of a rotational load.

Furthermore, consistent with some implementations, deflection portion 512 and/or cross member 524 of living hinge component 532 may be formed to provide a neutral bias to deflection portion 512. In this manner, although deflection portion 512 may be rotated upon exertion of pressure by needle 20, the dimensions and thicknesses of the relative components may be such that deflection portion 512 may be biased to return to its neutral position, thus maintaining a pressure on needle 20 throughout its range of motion.

As shown in FIG. 5, an inward facing groove 540 is formed in a central portion of cross member 538 and an outward facing groove 542 is formed in a portion of distal cross member 524 between deflection side rails 534 and 536. Grooves 540 and 542 are sized to accommodate a variety of puncture device gauge sizes. For example, similar to grooves 140/142 described above, one or both of grooves 540 and 542 may be v-grooves, although in other implementations, grooves 540/542 may have a different configurations, such as a cylindrical, slotted, etc.

As shown in FIGS. 5 and 6, during use, needle 20 may be inserted into grooves 540 and 542. To achieve a desired needle angle, an outward pivotal force may be applied to a proximal end of needle 20. This force causes needle 20 to urge cross member 538 outwardly relative to side rails 514 and 516, which causes deflection side rails 534 and 536 to rotationally deflect or deform relative to distal cross member 524.

As shown, the two grooves 540 and 542 are configured to guide needle 20 along the scan plane 14 of the probe 10. The grooves are oriented in opposing directions so that as pressure is applied by the needle 20 to rotate the deflection portion 512 about the live hinge to adjust the angle with respect to the fixed portion 510 of guide 500, the needle 20 is forced into both grooves simultaneously. Grooves 540/542 allow the puncture device guide to work with various sizes of puncture devices and hold them in the scan plane of the ultrasound probe while allowing the physician full control of the needle angle within the scan plane. FIGS. 5 and 6 illustrate needle 20 positioned within grooves 540/542 and oriented in a first (e.g., neutral or unflexed) angular position. As with puncture device guide 100 described above, puncture device guide 500 may also be formed as a single plastic or similarly flexible or resilient part, such as injection molded acetal, polypropylene, etc. In addition, consistent with puncture device guide 500 may be affixed to probe 10 or a probe cover in the manner described above in relation to FIG. 4.

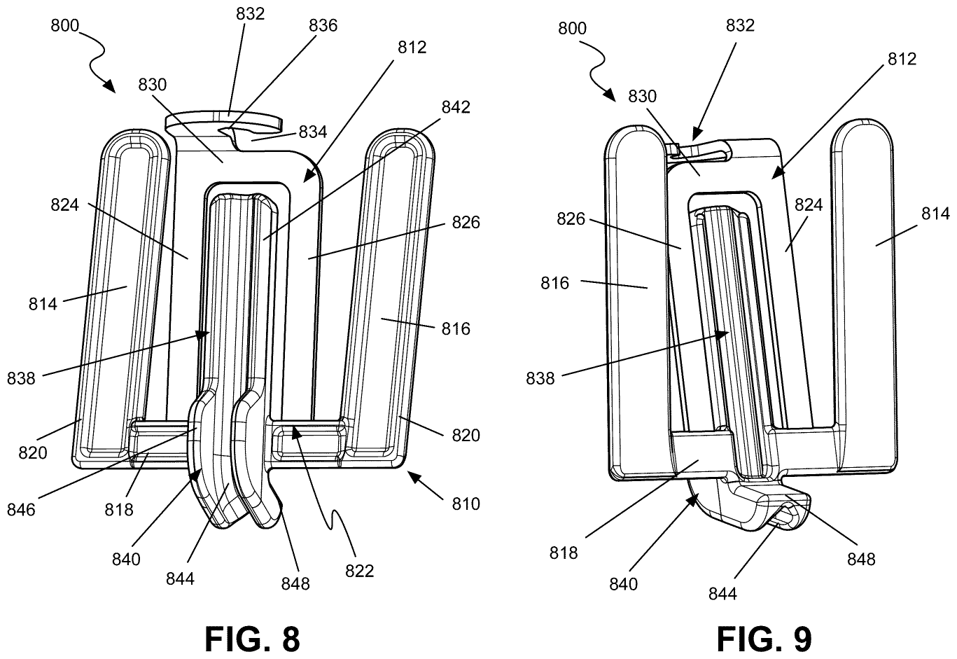

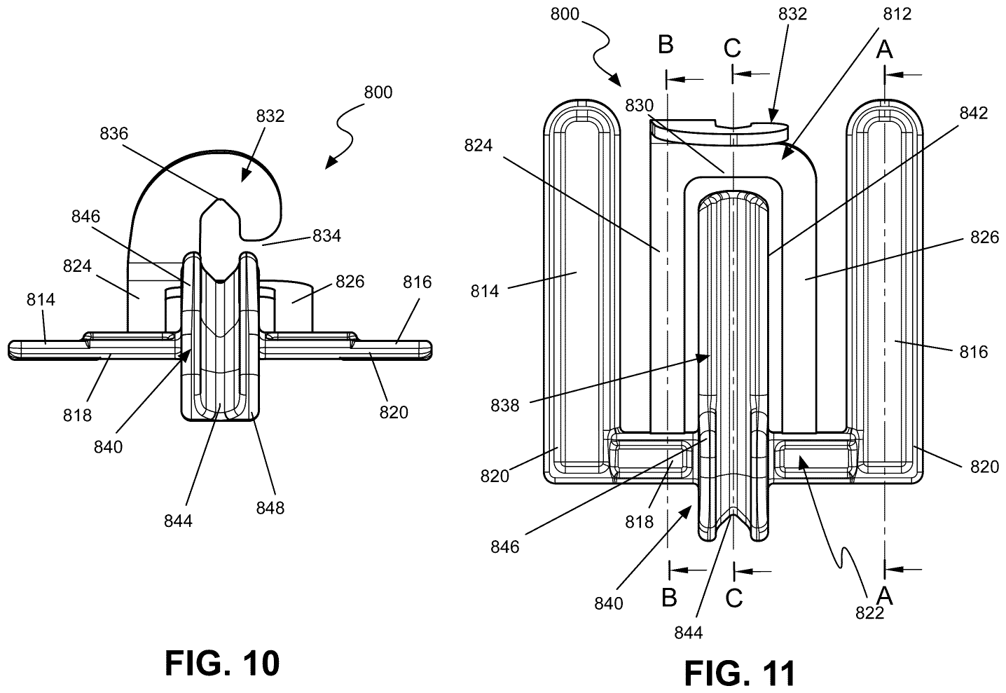

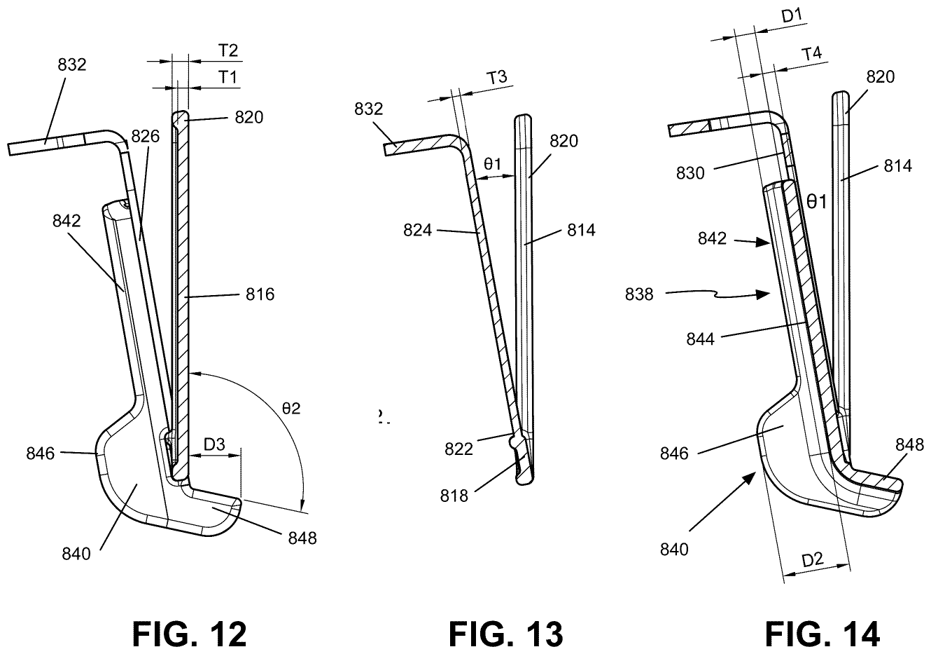

FIGS. 8, 9, 10, and 11 illustrate front isometric, rear isometric, bottom, and front views, respectively, of a yet another embodiment of a puncture device guide 800 consistent with implementations described herein. FIGS. 12, 13, and 14 illustrate cross-sectional views taken along the lines A-A, B-B, and C-C in FIG. 11, respectively. As shown, puncture device guide 800 includes a fixed portion 810 and a deflection portion 812 for supporting a puncture device (e.g., needle 20 described above) in a range of angular orientations relative to fixed portion 810.

Consistent with the illustrated embodiment, fixed portion 810 includes a U-shaped configuration having a pair of side rails 814 and 816 and a cross member 818 that connects side rails 814 and 816. In contrast to the embodiments of FIGS. 1-3 and 5-7, rather than including perpendicularly projecting legs, side rails 814/816 of puncture device guide 800 are generally planar elements configured to directly engage the ultrasound probe 10, a probe cover, or an adhesive mounting sheet, such as mounting sheet 110.

As shown in FIGS. 8 and 11, side rails 814 and 816 and cross member 818 each include a raised edge or lip 820 around at least a portion of their respective peripheries to increase rigidity or structure strength of these elements. As shown in FIG. 9, a rear surface of side rails 814 and 816 may be substantially flat. As shown in FIG. 12, central portion of side rails 814 and 816 may have a thickness T1, and lip 820 have a thickness T2, which may be approximately 50% thicker than T1. Exemplary dimensions of T1 and T2 are approximately 0.5 and 0.75 millimeters (mm), respectively.

Consistent with implementations described herein, cross member 818 may form the base of a living hinge component 822 for coupling deflection portion 812 to fixed portion 810. For example, deflection portion 812 may be formed with suitable dimensions relative to cross member 818 to enable deflection portion 812 to rotate or pivot about cross member 818 upon exertion of a rotational load.

Furthermore, consistent with some implementations, deflection portion 812 and/or cross member 818 of living hinge component 822 may be formed to provide a neutral bias to deflection portion 812. In this manner, although deflection portion 812 may be rotated outwardly upon exertion of pressure by needle 20, as described below, the dimensions and thicknesses of the relative components may be such that deflection portion 812 may be biased to return to its neutral position, thus maintaining a pressure on needle 20 throughout its range of motion.

As shown in FIGS. 8-10, deflection portion 812 comprises a pair of deflection side rails 824 and 826 that project proximally from cross member 818 parallel to side rails 814 and 816. Consistent with one implementation, and as shown in FIG. 13, deflection side rails 824/826 may project at a relaxed angular orientation θ1 relative to side rails 814/816. One exemplary angle θ1 may be approximately 10° although any suitable relaxed orientation in a range of approximately 0° to 15° may be provided.

A deflection cross member 830 connects the proximal ends of deflection side rails 824 and 826. In some implementations, deflection side rails 824 and 826 have a length shorter than that of side rails 814 and 816, so that deflection portion 812 is completely contained within fixed portion 810. As shown in FIG. 13, deflection side rails may have a thickness T3 sufficient to allow flexing of at least a portion thereof relative to cross member 818. An exemplary dimension of thickness T3 is approximately 0.35 mm.

Deflection cross member 830 includes a hook portion 832 that projects perpendicularly outwardly from deflection side rails 824/826. As shown, hook portion 832 includes an access opening 834 and an inwardly facing groove 836 accessible via the access opening 834. Groove 836 may be centered between deflection rails 824/826.

As shown in FIGS. 8-11, deflection portion 812 further includes a rigid guidance portion 838. Rigid guidance portion 838 projects both distally and proximally from cross member 818 between deflection side rails 824/826. As shown in FIG. 14, rigid guidance portion 838 is formed to have a similar angular orientation θ1 relative to side rails 814/816, as described above in relation to displacement side rails 824/826 in their relaxed state. Rigid guidance portion 838 includes a distal portion 840 and a proximal portion 842. Each of distal portion 840 and proximal portion 842 include an outwardly facing groove 844 formed in a central portion thereof and aligned with inwardly facing groove 836 in hook portion 832. As shown in FIG. 14, rigid guidance portion 838 may be formed of a thickness T4, which is greater than thickness T3 of deflection side rails 824/826. An exemplary dimension of thickness T4 is approximately 0.6 mm.

As shown in FIGS. 8 and 14, distal portion 840 of rigid guidance portion 838 may include groove walls 846 that project forwardly relative to proximal portion 842. As shown in FIG. 14, groove walls 846 provide groove 844 in distal portion 840 with an increased depth D2 relative to a depth D1 of groove 844 in proximal portion 842. Such an increased depth at distal portion 840 allows groove 844 to more fully support a puncture device through a variety of angular orientations, as described below.

In addition, consistent with implementations described herein, distal portion 840 of rigid guidance portion 838 includes a probe face engagement portion 848 that projects rearwardly relative to cross member 818. Probe face engagement portion 848 is configured to engage front face 11 of the ultrasound probe during installation, to ensure that puncture device guide 800 is properly positioned relative to the face. As shown in FIG. 12, in some implementations, probe face engagement portion 848 may be angled relative to side rails 814 and 816 by an angle θ2 to more closely conform to a front face of an ultrasound probe. One exemplary angle θ2 is approximately 100° although any angle corresponding to a radius of curvature or configuration of the front face of an ultrasound probe may be used, depending on the particular implementation.

During use, a puncture device may be inserted into grooves 836 and 844. To achieve a desired angle, an outward pivotal force may be applied to a proximal end of the puncture device. This force causes the puncture device to urge hook portion 832 outwardly relative to side rails 814 and 816, which causes deflection side rails 824 and 826 to rotationally deflect or deform relative to distal cross member 818.

As shown, the two grooves 836 and 844 are configured to guide a puncture device along a scan plane an ultrasound probe. The grooves are in opposing directions so that as pressure is applied by the puncture device to rotate the deflection portion 812 about the live hinge to adjust the angle with respect to the fixed portion 810. As discussed briefly above, in some embodiments, living hinge component 822 may include a spring-like configuration that urges deflection portion 812 to return to its neutral position throughout its range of motion. In such an implementation, groove 836 maintains a positive engagement with needle 20 throughout its rotation.

According to embodiments described herein, puncture device guide 800 may be formed as a single plastic or similarly flexible or resilient part. Exemplary manufacturing processes include injection molding the part out of acetal (polyoxymethylene), polypropylene, or any other suitable plastic material.

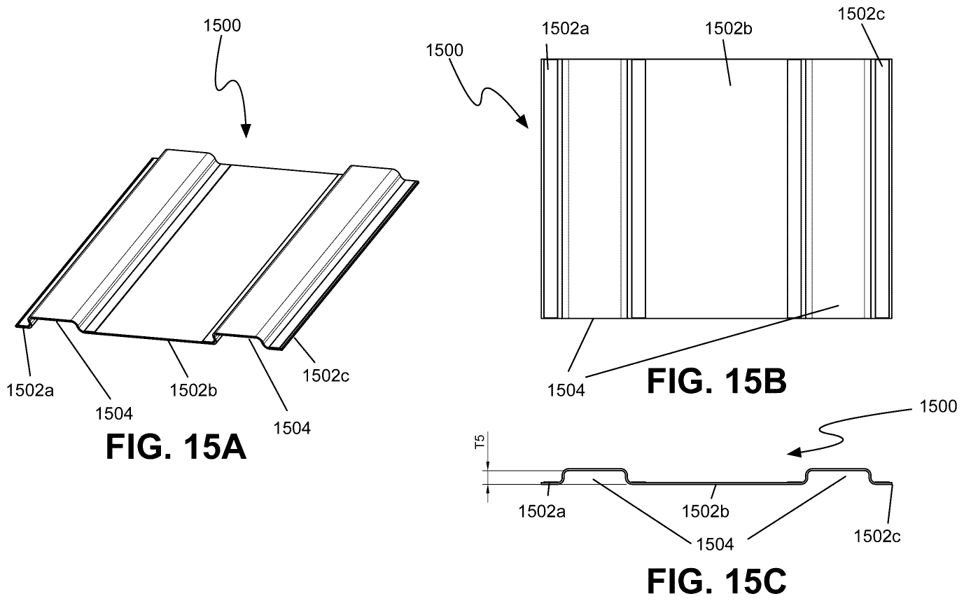

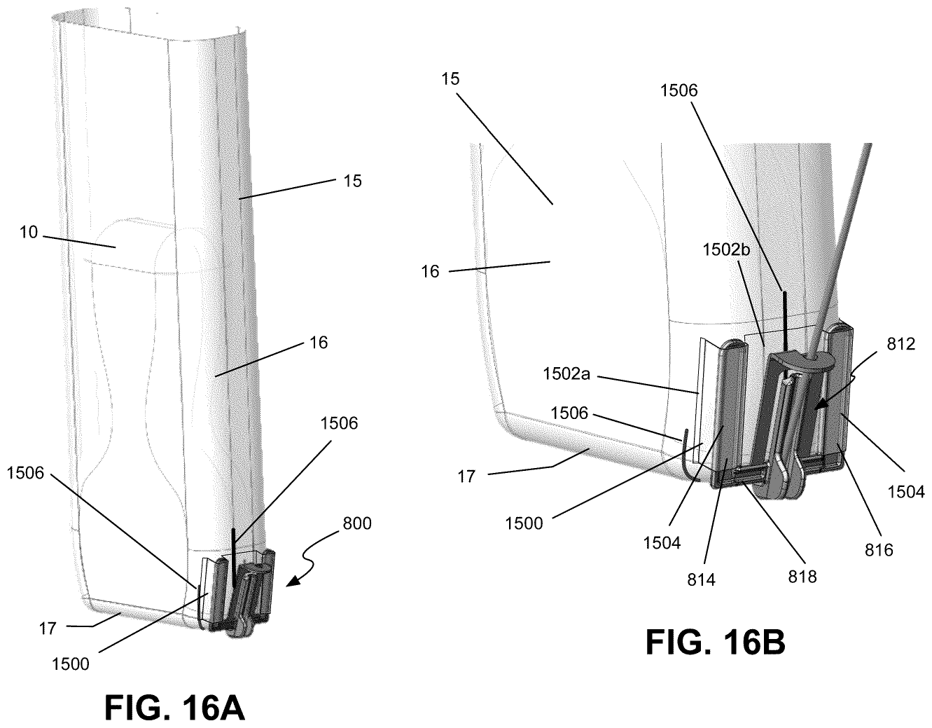

FIGS. 15A, 15B, and 15C illustrate front isometric, front, and bottom views, respectfully, of another embodiment of a mounting sleeve 1500 for use in securing puncture device guide 800 to an ultrasound probe 10. FIG. 16A is a front isometric view of mounting sleeve 1500 attached to a probe cover 15, which is installed onto ultrasound probe 10. In one implementation, probe cover 15 comprises a tubular structure 16 having a planar end portion 17 secured to form a closed end. In other implementations, probe cover 15 comprises a one-piece, sock-like, construction having a closed end and an open end. In any event, consistent with implementations described herein, mounting sleeve 1500 may be secured to the closed end, so as to provide an interface for receiving puncture device guide 800, as shown. FIG. 16B is a front isometric of the assembly of FIG. 16A, which illustrates a manner of coupling puncture device guide 800 to mounting sleeve 1500.

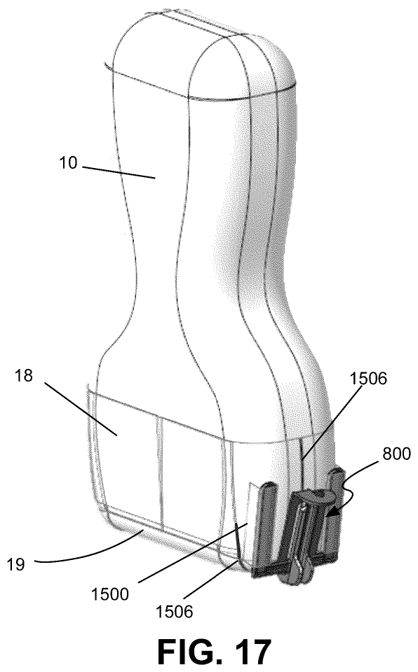

FIG. 17 is a front isometric of mounting sleeve 1500 attached to an ultrasound probe interface pad 18, which is installed onto ultrasound probe 10. As shown, ultrasound probe interface pad 18 includes a generally planar film having a patient-facing side 19 and a probe-facing side. Mounting sleeve 1500 is installed (e.g., welded, glued, etc. to the patient-facing side 19, so as to provide an interface for receiving puncture device guide 800, as shown.

Consistent with implementations described herein, mounting sleeve 1500 comprises a generally flat construction having adhesion portions 1502a, 1502b, and 1502c and puncture device guide receiving slots 1504. As shown in FIGS. 15A and 15C, puncture device guide receiving slots 1504 each have a relative spacing and thickness (denoted as T5) that generally corresponds to a thickness of side rails 814/816 including lip portions 820. In one implementation, mounting sleeve 1500 may be formed of a flexible polyurethane material, such as a similar material to that which forms the remainder of probe cover 15 or interface pad 18. In other implementations, a semi-rigid or rigid material may be used. Adhesion portions 1502a-1502c are adjacent puncture device guide receiving slots 1504 and have a generally planar configuration for engaging a surface of probe covering device, such as a probe cover 16 or a probe interface pad 17, as shown in FIGS. 16 and 17. Consistent with implementations described herein, adhesion portions 1502a-1502c may be permanently adhered to a predetermined location on probe cover 16 or a probe interface pad 17, such as via heat welding, a permanent adhesive, etc. Prior to use, side rails 814/816 may be slid into puncture device guide receiving slots 1504, such that probe face engagement portion 848 abuts front face 11 of the ultrasound probe (with probe cover 6 or probe interface pad 17 positioned therebetween).

Consistent with further implementations, probe cover 16 or probe interface pad 17 may be provided with an adhesive inner surface to secure the probe cover or the probe interface pad 17 to front face 11 prior to use.

As shown in FIGS. 16A, 16B, and 17, in some implementations, probe cover 16 and probe interface pad 17 may be provided with alignment stripes 1506 which may be used to assist the user in aligning both the mounting sleeve 1500 onto the probe cover or probe interface pad, as well as positioning the probe cover or probe interface pad with puncture device guide 800 pre-installed therein onto ultrasound probe 10. As shown, alignment stripes may include a vertical alignment stripe configured to align with a desired plane on the probe, such as the scan plane as shown in the figures. In other implementations, the vertical alignment stripe may be aligned with a central side plane of probe 10 to accommodate out-of-plane guidance. In some implementations, alignment stripes may also include a horizontal alignment stripe, which may assist the user in placing the probe cover/interface pad in a correct location on probe 10.

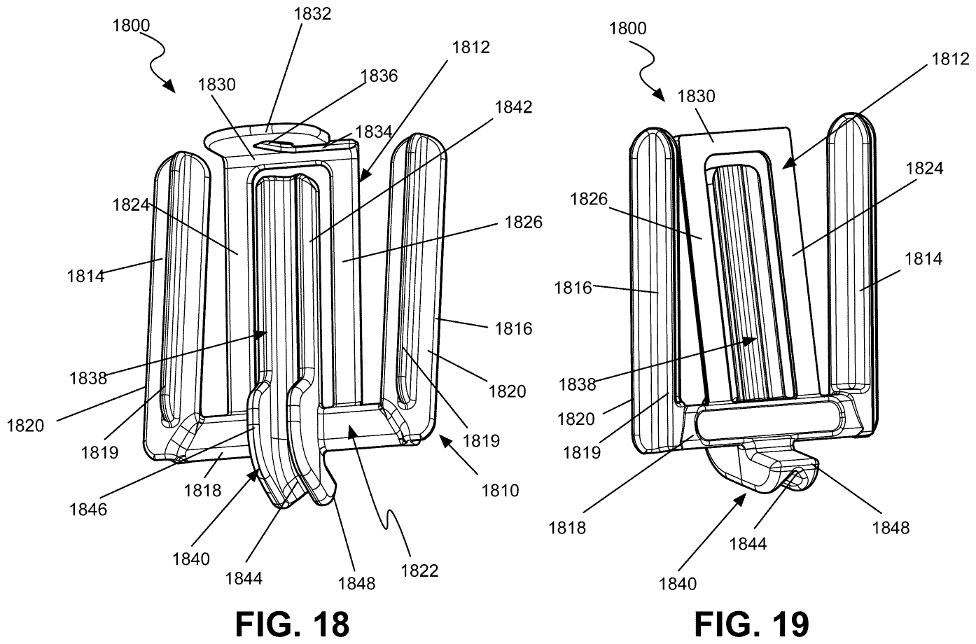

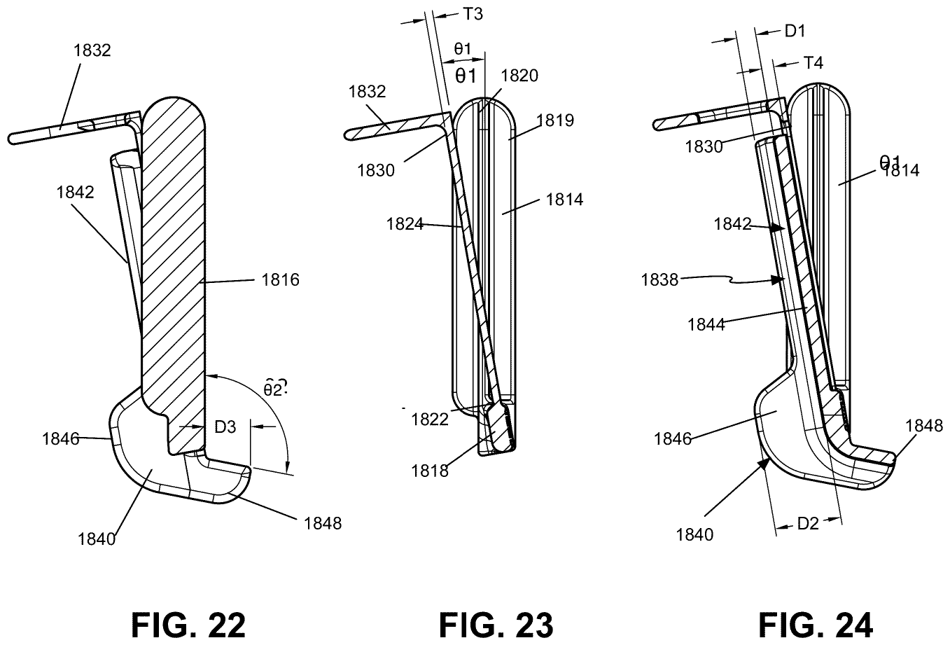

FIGS. 18, 19, 20, and 21 illustrate front isometric, rear isometric, bottom, and front views, respectively, of a yet another embodiment of a puncture device guide 1800 consistent with implementations described herein. FIGS. 22, 23, and 24 illustrate cross-sectional views taken along the lines A-A, B-B, and C-C in FIG. 21, respectively. As shown, puncture device guide 1800 includes a fixed portion 1810 and a deflection portion 1812 for supporting a puncture device (e.g., needle 20 described above) in a range of angular orientations relative to fixed portion 1810.

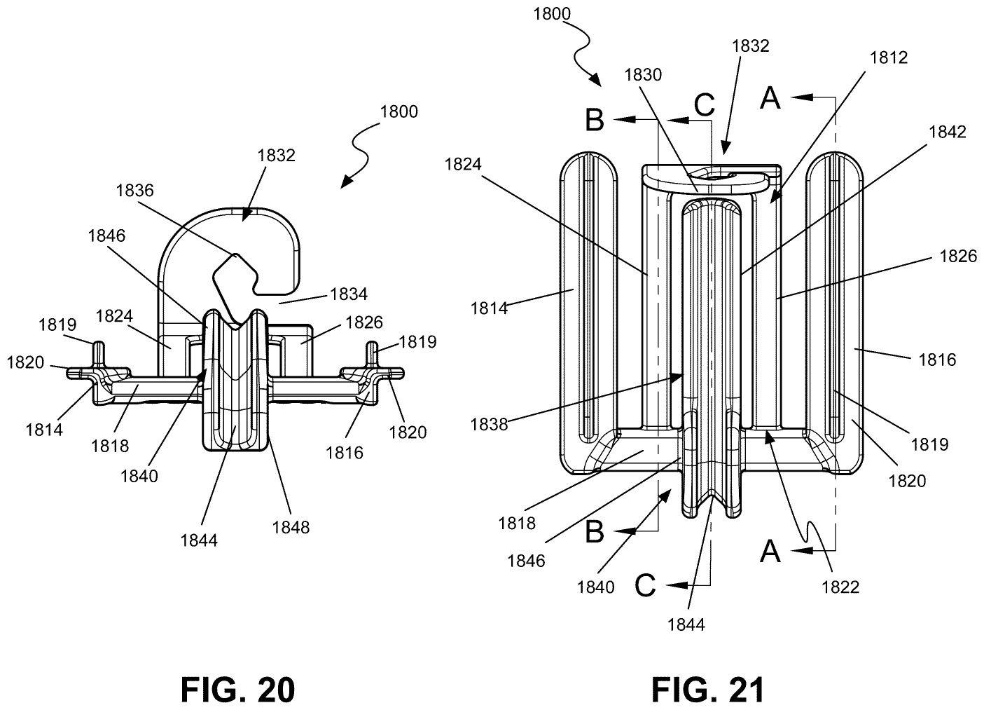

Consistent with the illustrated embodiment, fixed portion 1810 includes a U-shaped configuration having a pair of side rails 1814 and 1816 and a cross member 1818 that connects side rails 1814 and 1816. In contrast to the embodiments of FIGS. 8-14, rather than generally including flat side rails, side rails 1814/1816 of puncture device guide 1800 are three-dimensionally shaped to assist in securing side rails 1814/1816 within a mounting structure, 2500 described in detail below. In addition, a three-dimensional configuration may further provide increased strength and rigidity to side rails 1814/1816. For example, as shown most clearly in FIGS. 18 and 20, side rails 1814/1816 may include a cross or star configuration having opposing vertical ribs 1819 and opposing horizontal ribs 1820 oriented perpendicularly with respect to vertical ribs 1819. In one implementation, ribs 1819 and 1820 include a common or shared depth, such that an imaginary radial circumference of each side rail forms a circle having a diameter substantially similar to the depth of each rib. In the event that more or fewer than four ribs 1819/1820 are provided, such a common radial circumference may be maintained. As shown in the figures, side rails 1814/1816 may include rounded proximal ends 1822 to assist in installing or mounting puncture device guide 1800, as described below.

Consistent with implementations described herein, cross member 1818 may form the base of a living hinge component 1822 for coupling deflection portion 1812 to fixed portion 1810. For example, deflection portion 1812 may be formed with suitable dimensions relative to cross member 1818 to enable deflection portion 1812 to rotate or pivot about cross member 1818 upon exertion of a rotational load.

Furthermore, consistent with some implementations, deflection portion 1812 and/or cross member 1818 of living hinge component 1822 may be formed to provide a neutral bias to deflection portion 1812. In this manner, although deflection portion 1812 may be rotated outwardly upon exertion of pressure by needle 20, as described below, the dimensions and thicknesses of the relative components may be such that deflection portion 1812 may be biased to return to its neutral position, thus maintaining a pressure on needle 20 throughout its range of motion.

As shown in FIGS. 18-21, deflection portion 1812 comprises a pair of deflection side rails 1824 and 1826 that project proximally from cross member 1818 parallel to side rails 1814 and 1816. Consistent with one implementation, and as shown in FIG. 13, deflection side rails 1824/1826 may project at a relaxed angular orientation θ1 relative to side rails 1814/1816. One exemplary angle θ1 may be approximately 10° although any suitable relaxed orientation in the range of approximately 0° to 15° may be provided.

A deflection cross member 1830 connects the proximal ends of deflection side rails 1824 and 1826. In some implementations, deflection side rails 1824 and 1826 have a length shorter than that of side rails 1814 and 1816, so that deflection portion 1812 is completely contained within fixed portion 1810. As shown in FIG. 23, deflection side rails 1824/1826 may have a thickness T3 sufficient to allow flexing of at least a portion thereof relative to cross member 1818. An exemplary dimension of thickness T3 is approximately 0.35 mm.

Deflection cross member 1830 includes a hook portion 1832 that projects perpendicularly outwardly from deflection side rails 1824/1826. As shown, hook portion 1832 includes an access opening 1834 and an inwardly facing groove 1836 accessible via the access opening 1834. Groove 1836 may be centered between deflection rails 1824/1826.

As shown in FIGS. 18-21, deflection portion 1812 further includes a rigid guidance portion 1838. Rigid guidance portion 1838 projects both distally and proximally from cross member 1818 between deflection side rails 1824/1826. As shown in FIG. 24, rigid guidance portion 1838 is formed to have a similar angular orientation θ1 relative to side rails 1814/1816, as described above in relation to displacement side rails 1824/1826 in their relaxed state. Rigid guidance portion 1838 includes a distal portion 1840 and a proximal portion 1842. Each of distal portion 1840 and proximal portion 1842 include an outwardly facing groove 1844 formed in a central portion thereof and aligned with inwardly facing groove 1836 in hook portion 1832. As shown in FIG. 14, rigid guidance portion 1838 may be formed of a thickness T4, which is greater than thickness T3 of deflection side rails 1824/1826. An exemplary dimension of thickness T4 is approximately 0.6 mm.

As shown in FIGS. 18 and 24, distal portion 1840 of rigid guidance portion 1838 may include groove walls 1846 that project forwardly relative to proximal portion 1842. As shown in FIG. 24, groove walls 1846 provide groove 1844 in distal portion 1840 with an increased depth D2 relative to a depth D1 of groove 1844 in proximal portion 1842. Such an increased depth at distal portion 1840 allows groove 1844 to more fully support a puncture device through a variety of angular orientations, as described below.

In addition, consistent with implementations described herein, distal portion 1840 of rigid guidance portion 1838 includes a probe face engagement portion 1848 that projects rearwardly relative to cross member 1818. Probe face engagement portion 1848 is configured to engage a front face of the ultrasound probe during installation, to ensure that puncture device guide 1800 is properly positioned relative to the face. As shown in FIG. 22, in some implementations, probe face engagement portion 1848 may be angled relative to side rails 1814 and 1816 by an angle θ2 to more closely conform to a front face of an ultrasound probe. One exemplary angle θ2 is approximately 100° although any angle corresponding to a radius of curvature or configuration of the front face of an ultrasound probe may be used, depending on the particular implementation.

During use, a puncture device may be inserted into grooves 1836 and 1844. To achieve a desired angle, an outward pivotal force may be applied to a proximal end of the puncture device. This force causes the puncture device to urge hook portion 1832 outwardly relative to side rails 1814 and 1816, which causes deflection side rails 1824 and 1826 to rotationally deflect or deform relative to distal cross member 1818.

As shown, the two grooves 1836 and 1844 are configured to guide a puncture device along a scan plane an ultrasound probe. The grooves are in opposing directions so that as pressure is applied by the puncture device to rotate the deflection portion 1812 about the live hinge to adjust the angle with respect to the fixed portion 1810.

According to embodiments described herein, puncture device guide 1800 may be formed as a single plastic or similarly flexible or resilient part. Exemplary manufacturing processes include injection molding the part out of acetal (polyoxymethylene), polypropylene, or any other suitable plastic material.

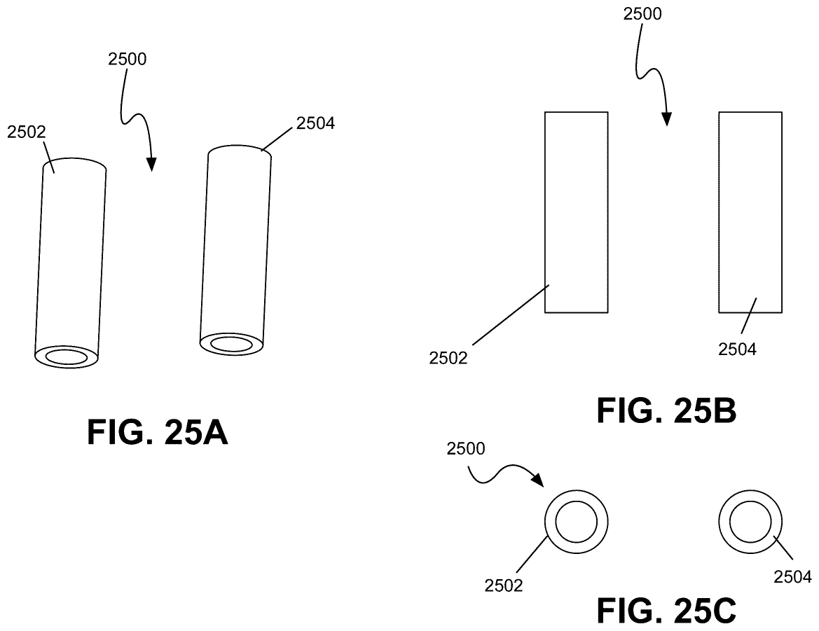

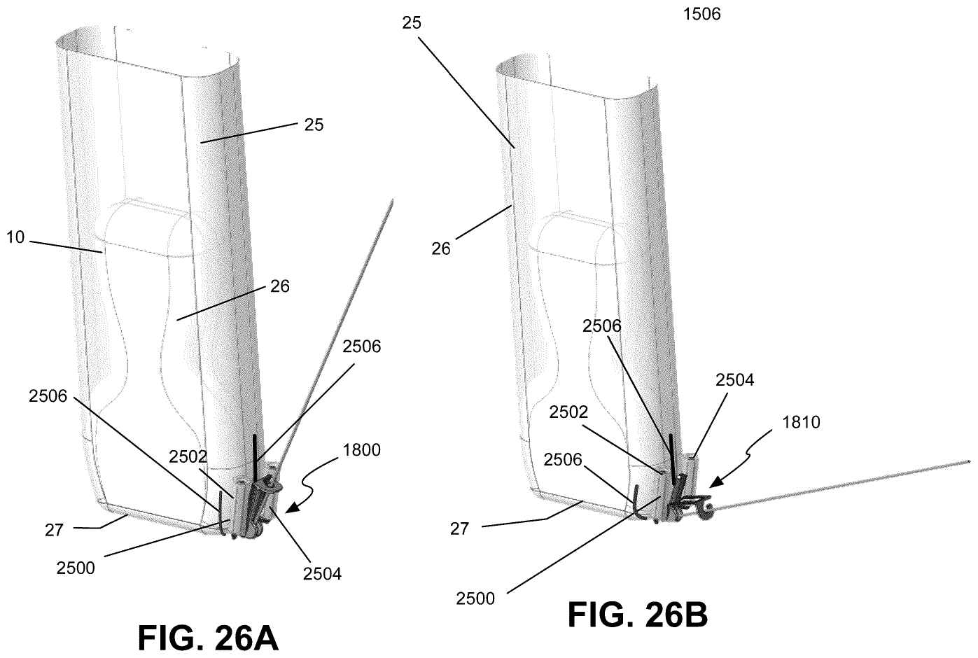

FIGS. 25A, 25B, and 25C illustrate front isometric, front, and bottom views, respectfully, of another embodiment of a mounting structure 2500 for use in securing puncture device guide 800 to an ultrasound probe 10. FIG. 26A is a front isometric of mounting structure 2500 installed onto a probe cover 25, which is installed onto ultrasound probe 10. In one implementation, probe cover 25 comprises a tubular structure 26 having a planar end portion 27 secured to form a closed end. In other implementations, probe cover 25 comprises a one-piece, sock-like, construction having a closed end and an open end. In any event, consistent with implementations described herein, mounting structure 2500 is secured to the closed end, so as to provide an interface for receiving puncture device guide 1800, as shown. FIG. 26B is a front isometric of the assembly of FIG. 26A, which illustrates a manner of coupling puncture device guide 1800 to mounting structure 2500.

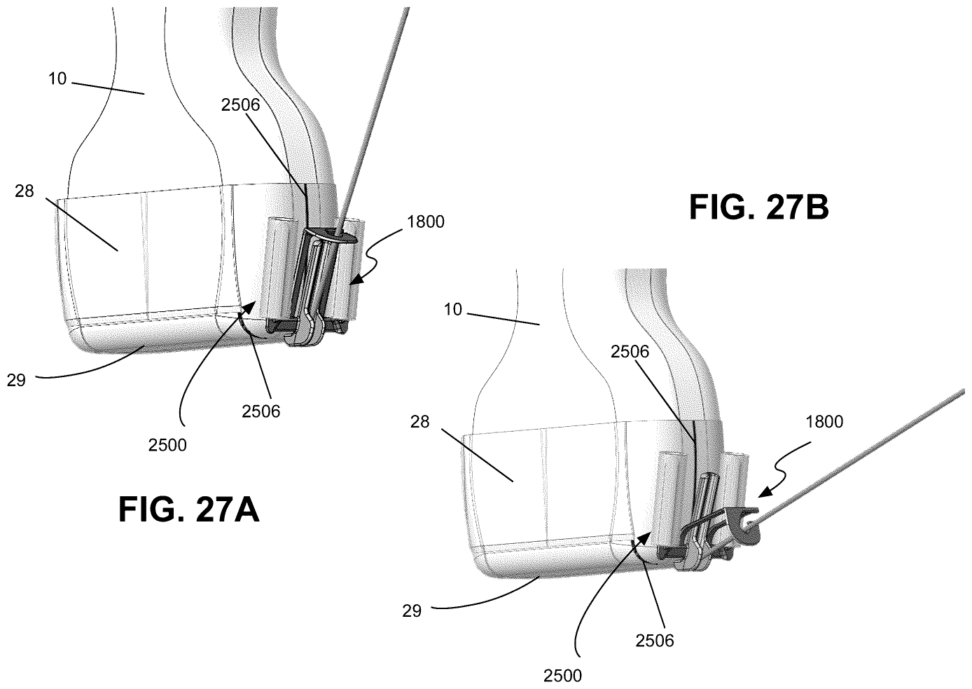

FIGS. 27A and 27B are front isometric views of mounting structure 2500 installed onto an ultrasound probe interface pad 28, which is installed onto ultrasound probe 10. As shown, ultrasound probe interface pad 28 includes a generally planar film having a patient-facing side 29 and a probe-facing side. Mounting structure 2500 is installed (e.g., welded, glued, etc. to the patient-facing side 29, so as to provide an interface for receiving puncture device guide 1800, as shown.

Consistent with implementations described herein, mounting structure 2500 comprises a pair of generally tubular side rail receiving elements 2502 and 2504. As shown in FIGS. 25A and 25C, side rail receiving elements 2502 and 2504 each have an inside diameter substantially similar to the height of ribs 1819/1820, such that side rails 1814/1816 may be received within side rail receiving elements 2502 and 2504. In one implementation, side rail receiving elements 2500 may be formed of a flexible polyurethane material, such as a similar material to that which forms the remainder of probe cover 25 or interface pad 28. In other implementations, a semi-rigid or rigid material may be used.

Consistent with implementations described herein, side rail receiving elements 2502 and 2504 may be permanently adhered to a predetermined location on probe cover 25 or a probe interface pad 28, such as via heat welding, a permanent adhesive, etc. Prior to use, slide rails 1814/1816 may be slid into side rail receiving elements 2502 and 2504, such that probe face engagement portion 1848 abuts front face 11 of the ultrasound probe (with probe cover 25 or probe interface pad 28 positioned therebetween). Consistent with further implementations, probe cover 25 or probe interface pad 28 may be provided with an adhesive inner surface to secure the probe cover or the probe interface pad 25/28 to front face 11 prior to use.

As shown in FIGS. 26A, 26B, 27A, and 27B, in some implementations, probe cover 25 and probe interface pad 28 may be provided with alignment stripes 2506 which may be used to assist the user in aligning both the mounting structure 2500 onto the probe cover or probe interface pad, as well as positioning the probe cover or probe interface pad with puncture device guide 1800 pre-installed therein onto ultrasound probe 10. As shown, alignment stripes 2506 may include a vertical alignment stripe configured to align with a desired plane on the probe, such as the scan plane as shown in the figures. In other implementations, the vertical alignment stripe may be aligned with a central side plane of probe 10 to accommodate out-of-plane puncture device guidance. In some implementations, alignment stripes 2506 may also include a horizontal alignment stripe, which may assist the user in placing the probe cover/interface pad in a correct location on probe 10.

The foregoing description of exemplary implementations provides illustration and description but is not intended to be exhaustive or to limit the embodiments described herein to the precise form disclosed. Modifications and variations are possible in light of the above teachings or may be acquired from practice of the embodiments.

Although the invention has been described in detail above, it is expressly understood that it will be apparent to persons skilled in the relevant art that the invention may be modified without departing from the spirit of the invention. Various changes of form, design, or arrangement may be made to the invention without departing from the spirit and scope of the invention. Therefore, the above-mentioned description is to be considered exemplary, rather than limiting, and the true scope of the invention is that defined in the following claims. Although the above-described puncture device guide devices include noted combinations of features, the inclusion of every feature need not be incorporated into each embodiment. For example, although puncture device guide devices 100, 500, 800, and 1800 each include a living-hinge based configuration, other guidance structures may be employed without departing from the spirit of the invention, as claimed. implementations. In particular, other types of puncture device guidance structures may be used, include fixed plates, multi-part pivoting components, etc.

No element, act, or instruction used in the description of the present application should be construed as critical or essential to the invention unless explicitly described as such. Also, as used herein, the article “a” is intended to include one or more items. Further, the phrase “based on” is intended to mean “based, at least in part, on” unless explicitly stated otherwise.

Use of ordinal terms such as “first,” “second,” “third,” etc., in the claims to modify a claim element does not by itself connote any priority, precedence, or order of one claim element over another, the temporal order in which acts of a method are performed, the temporal order in which instructions executed by a device are performed, etc., but are used merely as labels to distinguish one claim element having a certain name from another element having a same name (but for use of the ordinal term) to distinguish the claim elements.

What is claimed is:

1. A puncture device guide for use with an ultrasound probe having a probe body and a probe face arranged at a distal end of the probe body, the ultrasound probe face being configured to be placed proximate the outer surface of a patient; the ultrasound probe forming a scan plane perpendicular to said probe face, the puncture device guide comprising:
a fixed portion configured to attach directly or indirectly to the probe body; and
a deflection portion connected to the fixed portion by at least one spring or living hinge,
wherein the deflection portion comprises at least one groove configured to support a puncture device therein, and
wherein outward pressure on the at least one groove causes the deflection portion to rotate outwardly relative to the fixed portion to support the puncture device throughout a range of angles.

2. The puncture device guide of claim 1, wherein said fixed portion and said deflection portion are manufactured as a single part.

3. The puncture device guide of claim 1, wherein said fixed portion and said rotatable portion are formed of a flexible material.

4. The puncture device guide of claim 1, wherein the fixed portion comprises a first side rail and a second side rail joined to the first side rail by a cross member.

5. The puncture device guide of claim 4, wherein the deflection portion comprises at least one flexible member that projects from the cross member.

6. The puncture device guide of claim 5,
wherein the first side rail and the second side rail project perpendicularly from opposing ends of the cross member; and
wherein the flexible member of the deflection portion projects between the first and second side rails.

7. The puncture device guide of claim 6,
wherein the flexible member comprises a first deflection side rail and a second deflection side rail joined to the first deflection side rail by a deflection cross member.

8. The puncture device guide of claim 7,
wherein the at least one groove comprises:
a first groove in the cross member configured to face outward from the probe body when installed; and
a second groove formed in the deflection cross member configured to face inward from the probe body when installed,
wherein the first groove is aligned with the second groove.

9. The puncture device guide of claim 7,
wherein the deflection cross member comprises a hook portion adjacent the second groove which provides an access opening to the second groove to allow the puncture device guide to be removed from a puncture device inserted in the first and second grooves without requiring withdrawal of the puncture device.

10. The puncture device guide of claim 7, wherein the flexible member projects at a relaxed angular orientation relative to the first and second side rails.

11. The puncture device guide of claim 10, wherein the relaxed angular orientation comprises an angular orientation in the range of approximately 0° to 15°.

12. The puncture device guide of claim 7, further comprising:
a rigid guidance portion projecting from the cross member between the first deflection side rail and the second deflection side rails,
wherein the at least one groove comprises:
a first, outwardly facing groove formed in the rigid guidance portion.

13. The puncture device guide of claim 12, wherein the rigid guidance portion projects at an angular orientation relative to the first and second side rails.

14. The puncture device guide of claim 12,
wherein the rigid guidance portion includes a distal portion the projects distally from the cross member, and a proximal portion that projects proximally from the cross member,
wherein the rigid guidance portion includes groove walls, and
wherein the groove walls in the proximal portion have an increased depth relative to the groove walls in the distal portion.

15. The puncture device guide of claim 14, wherein the proximal portion of the rigid guidance portion projects rearwardly relative to the cross member to form a probe face engagement portion configured to engage a front face of the ultrasound probe during installation.

16. The puncture device guide of claim 15, wherein the probe face engagement portion is angled relative to the first and second side rails to conform to the front face of the ultrasound probe.

17. The puncture device guide of claim 5,
wherein the first and second side rails include a planar configuration.

18. The puncture device guide of claim 17, wherein the first and second side rails are configured to adhere to one of the ultrasound probe body or a sterile cover placed over the ultrasound probe body via an adhesive.

19. The puncture device guide of claim 17, wherein the first and second side rails are configured to be received within corresponding slots in a puncture device guide mounting sleeve coupled to or integrated with the ultrasound probe body or a sterile cover placed over the ultrasound probe body.

20. The puncture device guide of claim 5,
wherein the first and second side rails include a non-planar, three-dimensional configuration, and
wherein the first and second side rails are configured to be received within corresponding side rail receiving elements in a puncture device guide mounting structure coupled to or integrated with the ultrasound probe body or a sterile cover placed over the ultrasound probe body.

21. A puncture device guide system, comprising:
an ultrasound probe cover comprising:
a tubular portion having a first end and a second end; and
a planar portion secured to the first end of the tubular portion to close the first end of the tubular portion; and
a puncture device guide mounting structure secured to the planar portion;
a puncture device guide comprising:
a fixed portion configured to attach directly or indirectly to the probe body; and
a deflection portion connected to the fixed portion by at least one living hinge or spring,
wherein the fixed portion is configured to be attached to the puncture device guide mounting structure during use,
wherein the deflection member comprises at least one groove configured to support a puncture device therein, and
wherein outward pressure on the at least one groove causes the deflection portion to rotate outwardly relative to the fixed portion to support the puncture device throughout a range of angles.

22. The puncture device guide system of claim 21, wherein an inside surface of the planar portion comprises an adhesive layer for facilitating securing of the probe cover to an ultrasound probe.

23. A puncture device guide system, comprising:
an ultrasound probe interface pad comprising:
a probe-facing surface and patient facing surface; and
a puncture device guide mounting structure secured to the patient facing surface;
a puncture device guide comprising:
a fixed portion configured to attach directly or indirectly to the probe body; and
a deflection portion connected to the fixed portion by at least one spring or living hinge,
wherein the fixed portion is configured to be attached to the puncture device guide mounting structure during use,
wherein the deflection portion comprises at least one groove configured to support a puncture device therein, and
wherein outward pressure on the at least one groove causes the deflection portion to rotate outwardly relative to the fixed portion to support the puncture device throughout a range of angles.
